(19) United States
(12) Patent Application Publication
(10) Pub. No.: US 2025/0101366 A1
(43) Pub. Date: Mar. 27, 2025

(54) SYSTEM AND METHOD FOR SELECTING AND INTENSIFYING FERMENTATION IN BIOREACTORS

(71) Applicants: SUEZ INTERNATIONAL, Paris La Defense Cedex (FR); NEWhub Holding Company, Herndon, VA (US)

(72) Inventors: Mathieu HADDAD, Sevres (FR); Sudhir MURTHY, Herndon, VA (US); Nicolas GIRALDO-WINGLER, Robbinsville, NJ (US); Eugenio GIRALDO, Robbinsville, NJ (US)

(73) Assignees: SUEZ INTERNATIONAL, Paris La Defense Cedex (FR); NEWhub Holding Company, Herndon, VA (US)

(21) Appl. No.: 18/832,239

(22) PCT Filed: Jan. 20, 2023
(86) PCT No.: PCT/EP2023/051440

§ 371 Date: Jul. 23, 2024

Related U.S. Application Data
(60) Provisional application No. 63/413,734, filed on Oct. 6, 2022.
(60) Provisional application No. 63/302,117, filed on Jan. 23, 2022.

(51) Int. Cl. C12M 1/00; C12M 1/34

(52) U.S. Cl. CPC C12M 47/10; C12M 29/14; C12M 29/18; C12M 41/12; C12M 41/26; C12M 41/40; C12M 41/44; C12M 47/18

(57) ABSTRACT

Systems and methods for enhancing volatile fatty acid, ammonia or dihydrogen production in fermenting bioreactors are presented. Energy efficient evaporation methods and systems are disclosed. Integration of bioreactor and energy efficient evaporation enable use in existing bioreactors. Methods for further recovering fermentation products in bioreactors used for wastewater applications are also disclosed.

### TECHNICAL FIELD

The present disclosure is related to a system and method for treating a feed containing soluble or particulate carbonaceous material in a bioreactor. In particular, the disclosed embodiments drives the selection or intensification of carbonaceous material breakdown, fermentation or acetate oxidation in bioreactors. More specifically, the system and method of the present disclosure can enhance volatiles (including but not limited to volatile fatty acids, NH₃ or H₂) production and recovery from a bioreactor, especially in a fermenting bioreactor, by physical or chemical selection of reactions that drive their formation (including the selection of associated organisms) and their subsequent removal.

### BACKGROUND

Bioreactors are widely used in industry to conduct chemical transformations in a liquid broth using microorganisms and optionally their associated enzymes as catalysts. A feed broth is added to the bioreactor where microorganisms are present, the microorganisms conduct the desired chemical transformation, forming a treated broth and the treated broth is removed after remaining an appropriate time in the bioreactor. Bioreactors are widely used in the fermentation (including, and not limited to, food processing and pharmaceuticals) industry and in the wastewater treatment industry. In the latter, the feed broth is often municipal or industrial wastewater, or slurry fluids called sludge that contains a significant fraction of particulate matter. This particulate matter are often large complex biopolymers that require breakdown into smaller molecules in a process called hydrolysis often conducted outside the microbial cell by exoenzymes produced by the microorganisms present in the bioreactor prior to final conversion inside the microbial cell. In some instances, hydrolytic enzymes are added to the bioreactor to further accelerate particulate processing. In other cases, the bioreactors are maintained under favorable conditions to enhance such hydrolysis. Bioreactors that process particulate or soluble matter are usually completely mixed stirred reactors where the hydraulic retention time, HRT, and the solids retention time, SRT, are the same. Other reactors, including plug flow, batch, as well as sequenced batch reactor, are possible for the purpose of present disclosure. The hydraulic retention time is the average time that the fluid remains in the bioreactor, while the solids retention time is the average time that the particulate solids, including the microorganisms and enzymes responsible for the conversions occurring in the bioreactor, remains in the bioreactor. It is desirable to uncouple the HRT and the SRT in bioreactors because it enables intensification of the process processing the liquid at shorter retention times than the solids. Even though it is desirable to have this separation, it has proven elusive for bioreactors processing particulate substrates because there is no easy way of differentiating between the particulates, the microorganisms, the soluble substrates and the enzymes responsible for hydrolysis of particulates. Hydrolysis of particulates is considered the rate limiting step in bioreactors and, as such, concentrating the hydrolytic enzymes and microorganisms to enhance hydrolysis independently of the liquid in the bioreactor would yield a significant enhancement in the production of the bioreactor.

In addition, depending on the pH, hydrolysis products, such as volatile fatty acids (VFA), ammonia (NH₃), dihydrogen (H₂) can be inhibitory (such as, but not limited to, feedback inhibition) to some microorganisms and/or enzymes. For example, VFA become increasingly inhibitory as the pH is decreased, while NH₃ becomes increasingly inhibitory as the pH increases.

There is therefore a need for a system and method enhancing hydrolysis or breakdown of carbonaceous material in bioreactors, especially with reduced and/or controlled energy consumption.

### SUMMARY

The present disclosure presents an efficient way of improving the hydrolysis treatment of a feed containing particulate carbonaceous material. In particular, the disclosed embodiments allows uncoupling the HRT and the SRT of the active components involved in the hydrolysis of particulates in a bioreactor, especially by means of microorganisms and enzymes, also designed hereafter respectively as hydrolytic microorganisms and hydrolytic enzymes. Such active components include microorganisms and enzymes, involved in the hydrolysis and/or inhibitory products produced by the hydrolysis.

According to the present disclosure, a portion of the broth contained in the bioreactor is removed and at least in part evaporated to form a concentrate and an evaporate. The portion removed from the bioreactor contains microorganisms and hydrolysis products among which volatile inhibitory compounds are present. The evaporation of a part of the portion allows forming an evaporate containing at least a part of the volatile inhibitory compounds initially contained in said removed portion, while the concentrate contains at least the microorganisms initially contained in said removed portion. The inhibitory compounds are typically unionized, smaller product (or intermediates) molecules (such as VFA, dihydrogen, ammonia, methane, alcohol, aldehydes or ketones) and thus also easily and favorably subject to evaporation. Such a concentrate thus contains a reduced amount of the volatile inhibitory compounds initially contained in said removed portion. By returning at least a fraction of the concentrate into the bioreactor and collecting the evaporate, the accumulation of inhibitory compounds in the bioreactor is reduced, improving the hydrolysis, and the HRT and the SRT of the active components are uncoupled. The driving force of the reaction is the nature and concentration of the volatile compound(s), the temperature and the pressure (including negative pressure) in the evaporator. These features are being modulated in the present disclosure in a novel apparatus and system that, as needed, combines the formation of the volatile with the removal of the volatile through its physical or chemical selection supported by reactions considered in Henry's Law. The associated evaporation uncouples the solids residence time and the hydraulic retention time of the fermentation products (ammonia, H₂, VFA) in the bioreactor with respect to the other products.

Hydrolytic enzymes and microorganisms are indeed large molecules, non-volatile, that remain in solution when evaporation takes place. Evaporating a fraction of the water in the bioreactor would therefore selectively remove volatile molecules, including water and inhibitory hydrolysis products, and retain non-volatile molecules in the bioreactor.

By providing such uncoupling of the HRT and the SRT, the present disclosure allows intensifying hydrolysis or breakdown of particulates in bioreactors by concentrating the microorganisms/enzymes in the bioreactor and extracting from the bioreactor the inhibitory hydrolysis products.

Evaporation, however, is an energy intensive operation which is not easy to justify with the gains in intensification of the process. In one embodiment, at least a fraction of the evaporate may be condensed and the heat of condensation is recovered using approaches that include but are not limited to heat exchangers, heat pumps or vapor recompression, and for example for evaporation of a part of the portion removed from the bioreactor, the present disclosure further presents a way of recycling the energy needed to evaporate a fluid to significantly reduce the energy demand of evaporation of the process. Depending on the reaction and the evaporation that occurs, the products of a reaction can be included in the solid residence time within a reactor or in the evaporated fraction.

In one embodiment, the present disclosure further discloses the approach to condense and distill the evaporated volatiles by targeting a temperature or pressure for recovery of specific distillates, such as VFA (such as, but not limited to, acetic acid, propionic acids, butyric acids), dihydrogen, methane, ammonia, or any specific product or product groups intended for recovery. The heat of reaction and the heat of condensation can advantageously be used to manage the distillation approach. Recovery of multiple distillates is possible.

The management of ionized and unionized fractions (or alternatively protonated or unprotonated fractions through pH management) of hydrolysis or breakdown products are also envisioned within the context of reactions and the material being considered for evaporation, both in context of it's being harvested as a product, but also in context of its influence on any reactions occurring within the bioreactor or any downstream reactor.

Evaporating the contents of a bioreactor directly within the bioreactor presents several technological challenges, either the temperature of evaporation would be too high to be compatible with the microbial catalysts, enzymes and microbes, rendering them inactive; or a high vacuum would have to be applied directly in the bioreactor vessel to achieve practical rates of evaporation at low temperatures compatible with the biocatalysts. Applying vacuum directly to the bioreactor is thus possible at small scales, but once the scaling up of the process to large bioreactors takes place the structural demands on the vessel would be large and the costs of construction of such bioreactor vessel would be hard to justify compared to reduction in volumes due to intensification of the process. The disclosed embodiments also addresses this concern in addition to the high energy demand of evaporation.

### DESCRIPTION

According to a first aspect, the various disclosed embodiments concern a method for treating a feed containing carbonaceous material in a bioreactor, the method comprising:
a) hydrolyzing the feed in the bioreactor using microorganisms and enzymes,
b) removing a portion of the contents of the bioreactor, said removed portion containing microorganism, enzymes and hydrolysis products, the hydrolysis products containing volatile inhibitory compounds,
c) evaporating a part of said removed portion and forming an evaporate containing at least a part of the volatile inhibitory compounds initially contained in said removed portion, and a concentrate containing at least the microorganisms initially contained in said removed portion,
d) returning at least a fraction of said concentrate to the bioreactor and,
e) collecting said evaporate

The method of the present disclosure allows concentrating the microorganisms and/or enzymes within the bioreactor thus enhancing the hydrolysis of the particulates contained in the feed.

In one embodiment, the evaporate may contain at least one of volatile fatty acids, ammonia, dihydrogen and CO₂. Collecting the evaporate thus reliefs inhibition by either volatile fatty acids and/or dihydrogen and/or ammonia in the bioreactor by mechanisms of the removal of such volatiles or by shifting pH in the bioreactor to relieve such inhibition from the shifted pH or from the species being protonating or deprotonated as caused by shifting of pH. The change in pH is most often driven by the change in equilibria associated with the production or removal of acids and bases, although addition of chemicals is also possible.

In one embodiment, at least a fraction of the collected evaporate may be condensed forming a condensate and a non-condensable evaporate.

In this embodiment, (i) the non-condensable evaporate may be further treated to remove at least one substance selected from dihydrogen, ammonia, CO₂, and volatile fatty acids, or (ii) the condensate may be submitted to a distillation to recover at least one distillate, or both steps (i) and (ii) are possible. Advantageously, the treatment of the non-condensable evaporate may be a scrubbing treatment with an acid or a base or a solvent to further capture in a product stream containing non-condensable volatile substances, such as, but not limited to, VFA, ammonia, CO₂, and to recover a treated gas containing at least dihydrogen.

In addition, or alternatively, said condensate or said at least one distillate may be used as a raw material for a chemical battery (for attenuating peak demands) in, or for, a power or natural gas grid, biofuel, industrial or municipal or agricultural or household products, nutrient removal or recovery, disinfection or any other process.

In one embodiment, the heat of condensation may be recovered for further use, optionally for the evaporating step b) or for the hydrolysis step a), for example using a heat pump or a mechanical vapor recompression system or a heat exchanger. Preferably, the heat recovered is used for the hydrolysis step a).

The method of the present disclosure is performed while the bioreactor is operating. In other words, steps b)-e) are performed during hydrolysis step a).

In one embodiment, the bioreactor is a fermenting bioreactor.

In one embodiment, at least one parameter, selected from the pH and the oxidation-reduction potential in the bioreactor, may be controlled in step a):
by setting a setpoint range for said at least one parameter with a lower limit and an upper limit and activating steps b) to e) to maintain said at least one parameter within its setpoint range, or
by setting a setpoint value for said at least one parameter and continuously performing steps b) to e) to maintain said at least one parameter at its setpoint value, or
by setting activation periods at a specific activation frequency and activating steps b) to e) during a set duration at each activation period.

During activation of steps b)-e), one or several of the below controls (i) to (v) may be used to control said at least one parameter:
(i) adding at least one chemical selected from an acid, a base, an oxidant, a reductant,
(ii) controlling the removal frequency of the portion removed from the bioreactor in step b),
(iii) controlling the flow rate of the portion removed from the bioreactor in step b),
(iv) by controlling the temperature of the evaporation step c),
(v) controlling the pressure of the evaporation step c).

In one embodiment, the pH (such as, but not limited to, autogenously produced, or amended acids, alkali or buffers) in the bioreactor in step a) may be controlled to enhance the production of a specific hydrolysis product, such as, but not limited to, VFA, ammonia, dihydrogen, CO₂.

Advantageously, the pH in the bioreactor may be controlled at a pH value from 3 to 7, preferably from 3 to 6, more preferably from 4 to 6, most preferably from 5 to 6. Such pH control enhances VFA production.

Advantageously, the pH in the bioreactor may be controlled at a pH value from 8 to 10.5, preferably from 9 to 10. Such pH-control enhances ammonia production.

The redox-control allows selecting a specific biochemical reaction inside the bioreactor. Advantageously, the oxidation-reduction potential in the bioreactor may be controlled at a value from +350 mV to −400 mV. The upper limit of the redox potential value may be +350 mV, +300 mv, +250 mV, +200 mV, +100 mV or +50 mV. The lower limit of the redox potential value may be −400 mV, −350 mV, —300 mV, −250 mV. The redox potential may be controlled in a setpoint range or at a setpoint value within any of the previous lower and upper limits depending on the biochemical reaction considered.

In one embodiment, the method of the present disclosure may further comprise a step of providing a feed containing carbonaceous material comprising a thermal treatment sub-step and submitting said feed to the hydrolysis step a). The presence of a thermal treatment sub-step allows lowering the viscosity of a feed and increasing its dry matter content, and thus reducing the size of the vessel (less water in the contained volume), making its mixing, pumping or transfer easier (less thixotropic), and increase the VFA and/or ammonia and/or H₂ production.

In one embodiment, the method of the present disclosure may further comprise submitting the effluent produced by the hydrolysis step a), and optionally the fraction of said concentrate not returned to the bioreactor, to a thermal treatment, and optionally to an anaerobic digestion process.

According to a second aspect, the embodiments disclosed herein concern a system for a feed containing carbonaceous material in a bioreactor, the system comprising: the bioreactor, a feed supply to the bioreactor, an evaporator, and pipes, valves, and pumps to fluidly connect the above-mentioned parts of the system, said bioreactor being fluidly connected to an inlet of the evaporator and said evaporator comprising a first outlet for a concentrate and a second outlet for an evaporate, the first outlet being fluidly connected to said bioreactor.

Such system is advantageously adapted to implement the method of the present disclosure.

In one embodiment, the system may further comprise a condenser, at least one source of cold fluid connected to the condenser, the condenser being fluidly connected to the second outlet of the evaporator, and the condenser comprising a first outlet for a condensate and a second outlet for a non-condensable fluid.

Advantageously, the system may further comprise at least one of the following features:
(i) a scrubber and a source of at least one chemical selected from an acid, a base and a solvent, fluidly connected to said scrubber, and said scrubber being fluidly connected to the second outlet of the condenser,
(ii) a distillation equipment connected to the first outlet of the condenser.

Advantageously, the system may further comprise heat recovery equipment selected from a heat pump, a heat exchanger, a mechanical vapor recompression system, said heat recovery equipment being connected to the condenser to recover the heat of condensation and connected to the bioreactor or to the evaporator to provide heat thereto, preferably to the bioreactor.

Advantageously, the system may further comprise at least one parameter-control subsystem for controlling at least one parameter of the bioreactor selected from the pH and the oxidation-reduction potential in the bioreactor, said subsystem being configured to:
set a setpoint range for said at least one parameter with a lower limit and an upper limit and perform the following commands to maintain said at least one parameter within its setpoint range:
C1) removing a portion of the contents of the bioreactor and introducing said removed portion into the evaporator, said removed portion containing microorganisms, enzymes and hydrolysis products, the hydrolysis products containing volatile inhibitory compounds,
C2) evaporating a part of said removed portion to form an evaporate containing at least a part of the volatile inhibitory compounds initially contained in said removed portion, and a concentrate containing at least the microorganisms initially contained in said removed portion,
C3) returning at least a fraction of said concentrate to the bioreactor and,
C4) collecting said evaporate,
   or
set a setpoint value for said at least one parameter and continuously perform commands C1) to C4) to maintain said at least one parameter at its setpoint value,
   or
set activation periods at a specific activation frequency and perform commands C1) to C4) during a set duration at each activation period.

Advantageously, the parameter-control subsystem may be further configured to, during the performing of commands C1) to C4), control said at least one parameter in the bioreactor (i) by adding at least one chemical selected from an acid, a base, an oxidant and a reductant, (ii) by controlling the removal frequency of said portion removed from the bioreactor, (iii) by controlling the flow rate of said portion removed from the bioreactor, (iv) by controlling the temperature of the evaporator, (v) by controlling the pressure of the evaporator, or with a combination of one or several of (i) to (v).

In any of the described embodiments, the parameter-control subsystem typically comprises at least one parameter sensor to measure said at least one parameter inside the bioreactor, for example a pH sensor or a redox potential sensor or both, and one or more processors, for example microprocessors or microcontrollers, connected to the parameter sensor(s) and to appropriate valve(s) and/or pump(s) of the system. The parameter-control subsystem may also comprise one or more tanks containing a chemical (acid, base, oxidant, reductant), and pipes, valves and pumps to command the addition of the chemical. The parameter-control subsystem, more specifically the one or more processors, may be configured, in particular programmed, to implement commands C1)-C4) and/or steps b) to e) of the method of the present disclosure, in particular by implementing a feedback loop, and optionally to control said at least one parameter in the bioreactor by one or several of the previously described actions (i) to (v). Communication means, optionally bidirectional, may be provided between the parameter-control subsystem and the corresponding parameter-sensor and appropriate valve(s) and/or pump(s).

The separation of the evaporator from the bioreactor is a distinguishing feature of the disclosed embodiment. It provides for different materials of construction that may be needed for a typically larger bioreactor that may be under atmospheric or positive pressure from a typically smaller evaporator that may be under vacuum or negative pressure (with the bioreactor pressure as referential). Thus, the use of this approach with a fluid circulation loop between the two apparatuses provides for both construction economy and intensification. Indeed, a bioreactor can be any existing or reused concrete or steel tank at a facility with primary focus of biological reactions, while the evaporator is a functional tank that is used as a physical selector device for modulating the management of volatiles and water with underlying physical laws. The pH and oxidation-reduction potential-control subsystems are optional features that bring these two apparatuses together to control and bridge the reactions governed, one substantially by biology (in the bioreactor), and the other substantially by physics (in the evaporator). The physical driving force in the evaporator, thus can govern the selection of the biology and their preferred reactions in the bioreactor. The chemical controls from pH and oxidation-reduction potential management can also govern the selection of biology. This approach allows selecting a multitude and/or differentiated biological reactions in the bioreactor.

### DETAILED DESCRIPTION

### Definitions

The terminology used herein is for the purpose of describing particular embodiments only and is not intended to be limiting of the presently disclosed subject matter. While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. In describing the presently disclosed subject matter, it will be understood that a number of techniques and steps are disclosed. Each of these has individual benefit and each can also be used in conjunction with one or more, or in some cases all, of the other disclosed techniques.

Aspects and embodiments disclosed herein are not limited in application to the details of construction and the arrangement of components set forth in the description or illustrated in the drawings. Aspects and embodiments disclosed herein are capable of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

A primary clarification process, or primary treatment, generally reduces the solids and/or organic matter content of a wastewater to be treated. It is typically a settling stage, possibly assisted by prior addition of coagulant and/or flocculant, during which the wastewater is placed in a holding tank or settling tank. The solids contained in the wastewater settle to the bottom of the tank where they are collected. This stage produces so-called primary sludge and an effluent with a reduced solids content.

A secondary biological process, or secondary treatment, is a biological treatment in which the organic matter, nitrogen compounds and/or phosphorus compounds in the effluent from a primary treatment are assimilated or decomposed by aerobic and/or anaerobic and/or anoxic microorganisms. This secondary treatment also produces so-called biological sludge.

The term “aliquot” means a “portion” in the general meaning of “portion”. In the present specification, “aliquot” and “portion” are indifferently used.

In the present disclosure, a fermenting bioreactor is an aerobic, anoxic or anaerobic bioreactor where fermentation reactions take place forming products such as, but not limited to, VFA, alcohols, aldehydes, ketones, hydrogen gas (H₂), carbon dioxide.

Volatile inhibitory compounds are volatile compounds which have an inhibitory action and/or are toxic for microorganisms and/or enzymes contained in a bioreactor.

The term “evaporation” refers to the general process by which any substance is changed from a liquid state into a gaseous state. When the liquid contains water, the gas obtained may contain more or less steam depending on the evaporating conditions. As such, the term “evaporation” encompasses degassing where the gas phase is composed mostly of non-condensable products, optionally saturated with water. The term “evaporation” also encompasses processes where the gas phase is composed mostly of steam and non-condensable products.

The term “hydrolysis” or “breakdown” includes breakdown and/or solubilization of soluble or particulate carbonaceous material and/or macromolecules. The terms “hydrolysis” and “breakdown” may be used interchangeably.

The terms “redox” and “oxidation-reduction” are used interchangeably.

The words base and alkali are used interchangeably. The words hydrogen and dihydrogen are used interchangeably.

An acid, base, oxidant or reductant, can be in a solid, liquid, gas, ion, or in a charge form. The acid, base, oxidant and reductant are relative words and depend on the medium. A chemical for example can be an acid or a base depending on the pH of the medium (such as the evaporator or bioreactor contents). A chemical for example can be an oxidant or reductant depending on the oxidation-reduction potential state of the medium (such as the evaporator or bioreactor contents).

The expression “negative pressure” refers to the pressure of the evaporator with the bioreactor pressure as a reference. Such negative pressure thus corresponds to a pressure lower than the pressure of the bioreactor, the pressure of the bioreactor being usually maintained at atmospheric or positive pressure. A negative pressure may or may not be vacuum pressure, but likely vacuum pressure is typically operated at negative pressure.

A subsystem is a part of a system, and in the disclosed embodiments, not all subsystems are needed to comprise or fulfill the requirements of the embodiment or constitute the inventive step. Some subsystems are optional and are embodiments that may enhance the overall system. At least four subsystems are described: the bioreactor, the evaporator, the pH-control subsystem, and the redox-control subsystem. Smaller subsystems can be a timer control approach, etc. that are not explicitly called out but are nevertheless described in the specification.

The term evaporator refers to a tank or tanks, vessel or vessels, or, device or devices that can in part be used for evaporation reactions for removal of solutes or solvents. Water is a typical solvent and volatile compounds are typical solutes. Both reactions are governed by physical laws. The hydraulic retention time of an evaporator is often in the order of minutes or hours. The evaporator can be under negative or vacuum pressure, the term negative is a relative term governed by the pressure of the bioreactor as previously explained.

The term bioreactor refers to a tank or tanks, vessel or vessels, or, device or devices, that can be used to hold biology in a broth or in a fluid suspension, or in layers; in a batch, sequencing batch, or continuous process; that is completely mixed, intermittently mixed, or plug flow; or any combination of these configuration approaches. Other configurations are also possible to hold this biology. The hydraulic retention time of the bioreactor is often in the order of hours or days, for example from 1 to 8 days.

The dry solids content can be determined by means of APHA, 2005. Standard Methods for the Examination of Water and Wastewater. American Public Health Association, Washington, DC.

Class A type biosolids is a designation for dewatered and heated sewage sludge that meets U.S. EPA guidelines for land application with no restrictions. Class A biosolids meet the requirements established under 25 Pa. Code § 271.932(a) regarding pathogens and 25 Pa. Code § 271.933(b)(1)-(8) relating to vector attraction reduction Thus, class A biosolids are virtually free of pathogens can be legally used as fertilizer on farms, vegetable gardens, and can be sold to home gardeners as compost or fertilizer.

The recitation of numerical ranges by endpoints includes all integer numbers and, where appropriate, fractions subsumed within that range (e.g. 1 to 5 can include 1, 2, 3, 4 when referring to, for example, a number of elements, and can also include one, two or three significant digits such as 1.5, 2, 2.75 and 3.80, when referring to, for example, measurements). The recitation of endpoints also includes the recited endpoint values themselves (e.g. from 1.0 to 5.0 includes both 1.0 and 5.0). Any numerical range recited herein is intended to include all sub-ranges subsumed therein, including any value with one, two or three significant digits within specific ranges.

### Feed

The feed treated in the present disclosure contains carbonaceous material.

The feed may contain any material intended for fermentation reactions including by not limited to mining, agricultural, industrial or domestic derived material, including virgin or waste products from any process that produces this carbonaceous material, including organic fraction of municipal solid waste (OFMSW) and wastewater sludge.

The feed containing carbonaceous material may be, but not limited to, a sludge produced in a liquid treatment train in a wastewater treatment plant or any other sludge.

Said sludge could be, but not limited to, primary sludge from the underflow of a primary clarification process, or biological sludge from a secondary biological process in the treatment of wastewater, such as, but not limited to, waste activated sludge, or a combination of a primary sludge and a biological sludge.

The primary sludge may have been previously thickened, for example to a dry solids content of 2-15 m %.

The feed used in the embodiment can have been previously submitted one or several sub-steps selected from a thickening step, a screening step, a dewatering step, a dilution step. Those sub-steps may be part of a providing step, comprising a thermal treatment sub-step or not.

The feed used in the embodiment may present a concentration value of dry solids from 1 to 50 m %, preferably from 1 to 40 m % or from 1 à 35% m, for example from 2 to 15 m % or from 15 to 30 m %, or from 17 to 25 m %, or within any range defined by two of these limits.

The carbonaceous material may be in a dissolved, colloidal, or particulate form.

The feed also contains water. Any presence of water sufficient to drive biological reactions is considered suitable.

Non limiting examples of possible thickening equipments, screening equipments and dewatering equipments are given in the description of the figures.

### Optional Step of Providing a Feed Containing Carbonaceous Material

In one embodiment, the feed treated in step a) is issued from a providing step comprising a thermal treatment sub-step.

The thermal treatment sub-step can include a thermal hydrolysis process (THP) and/or a hydrothermal carbonization (HTC) process. In a THP process, a sludge, typically having a dry matter content of between 12% and 25% (mass %), are maintained at a temperature of between 140° C. and 170° C. typically during 30 minutes to 60 minutes. The HTC process typically operates at temperatures between 180° C. and 280° C. for a period of minutes to several hours in a non-oxidizing atmosphere.

Such providing step may be a thermal hydrolysis process.

Such providing step may further include one or several sub-steps selected from a thickening step, a screening step, a dewatering step, a dilution step, generally performed before the thermal hydrolysis sub-step.

### Hydrolysis Step a)

The hydrolysis step a) is performed in a bioreactor containing microorganism(s) and enzymes, such as a fermenting bioreactor or any other bioreactor in which breakdown of carbonaceous material occurs. The microorganism(s) may comprise, or consist of, a single strain or a consortium of microorganisms. The enzymes may be enzymes produced by the microorganisms present in the bioreactor and/or may be enzymes produced from other microorganisms and introduced into the bioreactor as external source of enzymes.

Hydrolysis step a) allows producing VFA, dihydrogen and ammonia among other hydrolysis products.

The bioreactor can be operated adding dioxygen as in an aerobic, or microaerophilic, digestion process, but can also be operated anaerobically, with no dioxygen addition, as in an anaerobic digestion, fermenter or reactor process. Yet, in other cases, dioxygen is provided but fermentation reactions still take place or are actually enhanced in the bioreactor. An example of an aerobic process with fermentation reactions, without limiting the application to said process, is the thermophilic aerobic digestion of sludges from wastewater treatment plants, also known by the acronym ATAD, from autothermal thermophilic aerobic digestion. In said bioreactor microorganisms grow decomposing the carbonaceous material present in the sludge and, in the case of an aerobic process, producing carbon dioxide and water, yet in an anaerobic digestion the microbial organisms work cooperatively to convert the particulate substrate present in the sludge into soluble substrates and in a series of reactions into methane and carbon dioxide. In aerobic and anaerobic bioreactors excess gases are released from the liquid product. Ammonia is also released during hydrolysis of proteins in the bioreactor and can become a significant process bottleneck. Similarly, products such as volatile fatty acids, VFA, (including acetic, propionic, butyric and higher acids), alcohols, aldehydes and ketones can also be released. The use of alternate electron acceptors/donors, or oxidants, or reductants to enhance or moderate fermentation rates or extent, using any of the above processes is explicitly disclosed.

As explained in reference to the evaporation step, a portion of the contents of the bioreactor is removed from the reactor and evaporated, and the concentrate is partly sent back to the bioreactor. The evaporation of the volatile products can affect a change in pH and/or oxidation-reduction potential change in the bioreactor thus allowing selecting and/or intensifying reactions inside the bioreactor.

The reactions occurring in the bioreactor to breakdown the carbonaceous materials contained in the feed are generally performed in acidic or basic conditions. Reactions performed in acidic conditions usually enhances the production of VFA among other hydrolysis products. Reactions performed in basic conditions usually enhances the production of NH₃ among other hydrolysis products.

Depending on the pH, the product of the reaction, such as unionized VFA and ammonia, can be inhibitory.

In the present disclosure, volatile inhibitory compounds thus include one or several of volatile fatty acids, ammonia, dihydrogen.

The removal of such toxic volatile compounds can alleviate such inhibition within the process/reactor or in downstream processes/reactors increasing conversion rates and improving production yields.

In addition, the monitoring of the redox potential allows determining the biological reaction(s) occurring inside the bioreactor. By setting the redox potential in or out the redox potential range of a specific biological reaction, it is thus possible to select specific biological reaction(s).

In one embodiment, at least one parameter selected from the pH and the oxidation-reduction potential in the bioreactor is controlled during step a), for example by means of a pH-control subsystem and/or an oxidation-reduction potential-control subsystem connected to the bioreactor to enhance specific reactions within the reactors. A single subsystem controlling both pH and oxidation-reduction potential is also possible.

In one embodiment, the bioreactor is a fermenter where the production of volatile fatty acids is preferred. High concentrations of volatile fatty acids and dihydrogen are observed in fermenters to the extent that pH is reduced, protonated volatile fatty acids form and inhibition of the production of volatile fatty acids is observed. Net removal of volatile fatty acids, dihydrogen and/or ammonia from the fermenter occurs in the cycle of portion removal, evaporation and return of concentrate as previously described. As a result of net acid removal, pH in the bioreactor would increase if the amount of said acid removed in the evaporation process is larger than the amount of acid biologically produced in the bioreactor. In this embodiment, the pH in the bioreactor may be controlled at a pH from 3 to 7, preferably from 3 to 6, more preferably from 4 to 6, most preferably from 5 to 6.

Addition of an acid might be necessary to operate the bioreactor at this low pH. Examples of acids that may be used include, without limitation, hydrochloric acid, organic acids and any acidic gas or liquid produced during the hydrolysis step a), typically recovered from the evaporation or condensation steps, such as CO₂-, or VFA-containing gas or liquid (where the CO₂ containing gas may be an acid or base depending on the initial pH).

In another embodiment, the bioreactor is operated at high pH and the production of NH₃ is preferred. Is this embodiment, the pH in the bioreactor may controlled at a pH from 8 to 10.5, preferably from 9 to 10. Addition of a base might be necessary to operate the bioreactor at this high pH, ammonia inhibition might limit the fermentation process and removal of ammonia in the evaporator will relief the inhibition while recovering ammonia simultaneously.

Examples of bases that may be used include, without limitations, potassium hydroxide, calcium hydroxide, sodium hydroxide, lime, magnesia, potash, and the carbonates or bicarbonates of sodium, magnesium, calcium and potassium, any salt of a strong base/weak acid, and any basic gas or liquid produced during the hydrolysis step a), typically recovered from the evaporation or condensation steps, such as CO₂-, or ammonia-containing gas or liquid.

In another embodiment, the VFA and ammonia extraction could occur in the same vessel or sequenced vessels, where for example, after sufficient removal of one volatile, the second volatile is removed. Here, for example, the process could start with an alkaline hydrolysis step (towards the pKa of ammonia) for the removal of unionized ammonia, and once sufficient ammonia is removed, the pH is allowed to drop (towards the pKa of VFA) for the removal of unionized VFA. The reverse approach is also possible, where the VFA extraction precedes the ammonia extraction. A volatile extract from a reaction can be used to drive the change in pH from high to low or low to high.

Other reactions that can be encouraged or discouraged by pH control are precipitation reactions (such as amorphous and crystalline substance, such as struvite, vivianite, hydroxyapatite, brushite, etc.) that are dependent on the type of evaporated solutes that can directly or indirectly (such as by changing pH, oxidation-reduction potential, or temperature) impact the solubility of a reaction and the yield of a precipitate. These reactions can be supported through the addition of iron, magnesium or calcium compounds.

The control of the pH may be performed by any of the setting previously described, for example as detailed in the description of FIG. 2, such description being applicable to any of the embodiments disclosed in the present specification.

The approach for managing a pH value, could be simultaneous (where the reaction increasing the pH is balanced by reaction decreasing the pH), sequential in space (for example, the transfer or interchange between the reactor and evaporator), or sequential in time (for example in a bandwidth control within a vessel).

Similarly, the removal of volatiles (especially those containing reducing or oxidizing equivalents) from the evaporator, can effect a change in oxidation-reduction potential in the bioreactor, that can be used with advantage to manage its control either in a simultaneous or sequential manner, its increase or decrease, as already described for pH.

In one embodiment, the bioreactor includes the provision of any alternate electron acceptor such as (including but not limited to) air or dioxygen, or nitrates, are augmented to favorably increase the hydrolysis rates in a transient (in space or time), bandwidth, or poised (simultaneous reactions) manner while optionally and simultaneously or sequentially controlling (through transient (in space or time), bandwidth, or poised oxidation-reduction potential management) unfavorable reactions (optionally such as aerobic metabolism or methanogenesis).

The oxidation-reduction potential in the bioreactor may be controlled at a value from +350 mV to −400 mV to control the production of VFA, dihydrogen, ammonia, or other volatile intermediates that can then be subject to evaporation in the subsequent step b). The man skilled in the art knows how choosing the redox potential value to select specific biochemical reaction(s).

Addition of an oxidant or a reductant might be necessary to maintain the redox potential at a setpoint value or within a setpoint range. Such oxidant or reductant may be liquid or gaseous.

Examples of oxidants that may be used include electron acceptors such as dioxygen, nitrate or ferric ions, air, including any gas or liquid produced during the hydrolysis step a) and containing an oxidant.

Examples of reductants that may be used include electron donors such as, but not limited to, dihydrogen, including any gas or liquid produced during the hydrolysis step a) and containing a reductant.

The oxidant/reductant addition may be performed using blowers or bubbleless systems (including, but not limited to, membrane aerated biofilms) or using electrical systems (such as, but not limited to, cathodic, anodic, electron beam, proton beam or electrolysis), where dioxygen or dihydrogen, when used, could be recycled, produced in situ, or sourced externally.

For example, the oxidation-reduction potential values may be maintained at a value somewhat higher (less negative) than the reactions supporting methanogenesis (such as any value greater than −50 mV) or lower than values that are typical for denitrification (such as any value less than 0 mV). The addition of electron acceptors such as dioxygen, nitrate or ferric ions, help increase or balance the oxidation-reduction potential conditions in a manner that hydrolysis rates are maximized, methanogenesis rates are minimized, while intermediate fermentation products are preferably generated. These oxidation-reduction potential values for control, can have a deviation from above (of 0 and −50 mV) mentioned oxidation-reduction potential values, by plus or minus 50 mV or as much as 200 mV. For example, the values could range from 150 mV for an upper value, to −250 mV for a lower value, or any value in between. The goal is to maximize both rate and extent of production of volatile compounds for evaporation. Each value is chosen to either increase or decrease a reaction rate or stoichiometry, and eventually to select or deselect an organism (including but not limited to, acetocalastic methanogens, hydrogen utilizing methanogens, acetate oxidizing organisms, sulfate reducers, fermenters or low redox bacteria of all types (including but not limited to acidogens, acetogens, methanol or ethanol producers), acetate oxidizers, sulfur oxidizers or sulfur reducers, iron oxidizers or iron reducers, facultative denitrifiers, or aerobic heterotrophs). While inhibition of methanogenesis is usually desired in such reactions and oxidation-reduction potentials involving hydrolysis and acetate oxidation, the subsequent promotion of additional acetate oxidation or methanogenesis (for example at redox values lower than −250 mV) is also possible and part of the selection process considered within the bounds of the disclosed embodiments.

Thus, inhibitory volatiles can be managed by adjustments of oxidation-reduction potential and/or pH through the step of evaporation. In an example, an oxidation-reduction potential setpoint range can be setup between the removal and production of hydrogen between the bioreactor and the evaporator.

A cyclic oxidation-reduction potential setpoint range can be setup between the oxidation and reduction of iron or sulfur (or any metal or non-metal) in the bioreactor or any other species that can undergo cyclic oxidation reduction associated with their valence state.

In one embodiment, the hydrolysis can be conducted at any mesophilic or thermophilic temperatures, the thermophilic temperatures are intended to either pasteurize pathogens (including Class A type biosolids) or indicators of pathogens (such as fecal coliforms or E. coli<1000 MPN/g), or to increase reaction rates. Alkaline conditions may also be possible either to increase reaction rates or to achieve favorable conditions supporting destruction of pathogens or their indicators.

The solid retention time of the hydrolysis step a) is typically from 1 to 8 days.

In another embodiment, the vector attraction reduction (VAR) as described by volatile solids (VS) destruction or removal can meet the goals of stabilization as described in any global regulation, including but not limited to United States, Canada, European Union, United Kingdom, Australia, New Zealand, India, China or South East Asia. The percent VS destruction or removal in the bioreactor can be between 10% and 60%, depending on the reaction rates and initial VS concentrations, and the VS remaining can be as low as 30% remaining, but more likely close to 50% remaining. The VAR can also be achieved through drying (such as increasing dry solids content greater than >50%) or by increasing pH such as greater than pH=10. A combination approach is also possible to manage VAR.

The effluent produced by the hydrolysis step a) in the bioreactor can be submitted to an optional thermal treatment, preferably followed by an anaerobic digestion process. The thermal treatment may be a THP process or a HTC process or a hydrothermal liquefaction (HTL) process or a pasteurization process or a combination of two or more of these processes. The THP process and HTC process are as previously described. The HTL process is typically performed at 200-320° C. under pressure (from 40 to 180 bar) in the presence of dioxygen or air. The pasteurization process is typically performed at 70° C. those processes are well known from the man skilled in the art and will not be further described.

The final anaerobic digestion process (which may be mesophilic or thermophilic) allows for:
energy recovery (finalizes the recovery of volatile solids, VS, and produce energy for the system to be energy neutral/positive)
proper dewatering as the sludge downstream of the bioreactor will still have a concentration of VFA that is likely to hinder polymer consumption/sludge conditioning and proper dewatering.

The hydrolysis step could occur on its own or in context of other processes including thermal processes (such as but not limited to thermal hydrolysis or hydrothermal carbonization), thickening processes, dewatering processes, precipitation or crystallization processes, digestion processes, drying (including but not limited to biodrying) processes, pyrolysis processes, or any process where co-conversions are beneficial.

### Evaporation Step

During this step some or all the volatile compounds contained in the portion removed from the bioreactor is evaporated.

The portion of the contents of the bioreactor can be removed from the bioreactor in step b) by any appropriate means including pipe(s), valve(s), pump(s).

Generally, in evaporation step c), part of the water and other volatile substances contained in said removed portion, such as, but not limited to, carbon dioxide, ammonia, volatile fatty acids, low molecular weight fermentation compounds, hydrogen sulfide or other odorous sulfur reduced compounds, are evaporated from the removed portion forming an evaporate, and reducing the volume of the removed portion forming a concentrate.

At least a fraction of the concentrate containing the biocatalysts (microorganisms and/or enzymes) is returned to the bioreactor where it is mixed with the contents of the bioreactor. The fraction of said concentrate not returned to the bioreactor can be submitted to an optional thermal treatment, preferably followed by an anaerobic digestion process. The thermal treatment may be a THP process or a HTC process or a HTL process or a pasteurization process, or a combination of two or more of these processes. In particular, the concentrate not returned to the bioreactor can be mixed with the effluent produced by the bioreactor before being submitted to the optional thermal treatment, and optional anaerobic digestion process.

The evaporate containing water vapor, ammonia, and other volatile substances, such as volatile fatty acids, is collected.

The evaporation step c) is typically performed at temperature and pressure selected to avoid damage of the microorganisms and/or enzymes contained in the portion to evaporate. The man skilled in the art knows how to choose a temperature and/or pressure that do not damage and/or degrade the microorganisms and/or enzymes depending on their nature.

Generally, the evaporation step c) is operated at a same temperature as the bioreactor or a temperature inferior to the temperature of the bioreactor.

Generally, the evaporation step c) is operated at a negative pressure with respect to the pressure inside the bioreactor, vacuum being one possibility of negative pressure operation. In other words, step c) is typically operated at a pressure inferior to the pressure inside the bioreactor.

When at the operating pressure, the temperature is above the boiling point of the removed portion (which boiling point generally corresponds to the water boiling point), evaporation step c) is a degassing step, where the evaporate is vapor composed mostly of non-condensables with little or no steam.

When at the operating pressure, the temperature is at or under the boiling point of the removed portion, step c) is an evaporation step, where the evaporate is vapor composed mostly of steam and non-condensables. As the pressure is lower than in the previous case, the quantity of non-condensables extracted is expected to be higher.

Typically, the temperature of the evaporation step c) is high as possible, but similar or lower than the bioreactor temperature, ideally in the thermophilic range, although the embodiment also applies to mesophilic bioreactors. Temperature may be within the range 45-75° C. when the bioreactor is operated in the thermophilic conditions and within the range of 20-45° C. when the bioreactor is operated in the mesophilic conditions.

Typically, the pressure of the evaporation step c) is between 33 and 800 millibars absolute pressure. The extent of evaporation of the solvent in the process is controlled during evaporation by controlling pressure of the evaporation in step c), this enables evaporating dissolved gasses in the removed portion while at the same time controlling the evaporation rate.

The evaporation step can be operated in an evaporator, external to the bioreactor, such as, but not limited to, a forced circulation evaporator, or a rising film or falling film evaporator, or an agitated thin film evaporator, or a multiple effect evaporator or a self-cleaning evaporator, or further a flash evaporator, also known to some as a flash cooler process, or others evident to someone skilled in the art. Such evaporator may act as a degassing tank when the operating temperature is above the boiling point of the removed portion, or as a boiler when the operating temperature is at or below the boiling point of the removed portion.

The evaporator is typically configured with a system to limit and control the evaporation pressure and temperature inside the evaporator, for instance, a vacuum generation equipment, such as a vacuum pump, connected to its headspace.

### Optional Condensation Step

In some embodiments, at least a fraction of the collected evaporate may be condensed forming a condensate and a non-condensable evaporate.

Typically, the evaporate temperature is reduced to the point where a fraction of the volatile molecules are condensed.

Much of the condensation that takes place is condensation of water vapor forming a condensate liquid. The condensate also contains other volatile substances that fractionally condense and dissolve in the liquid such as, but not limited to, ammonia or volatile fatty acids, carbonic acid or sulfur compounds. The condensate may be reused in the process for pH control and/or redox potential control of the bioreactor depending on its composition.

Such step is typically performed in a condenser which is connected to at least one source of cold fluid. The source of cold fluid can be a heat pump or any other suitable source of cold fluid, such as, but not limited to, a cooling tower, or a chiller. In some embodiments, the condenser can be of the type that enables recovery of the heat of condensation using an equipment such as, but not limited to, a heat exchanger, a heat pump, a mechanical vapor recompression system.

In some embodiments, when running the system with high evaporation rates (for example when step c) is operated at a temperature at or below the boiling point of the removed portion), it is advantageous to recover the heat of condensation of the evaporated fluid, in which case a mechanical vapor recompression unit (or any other equipment allowing such heat recovery, as listed above) may be integrated to condense the fluid vapor in the evaporate and return the heat of condensation to the bioreactor.

The heat of condensation recovered is preferably used as a source of heat for the bioreactor, although its use as a source of heat to the evaporator is also possible.

The evaporate that remains after passing through the condenser is the non-condensable evaporate.

Most often, the condensation step is not optional and the condenser forms with the evaporator a subsystem.

### Optional Treatment of the Non-Condensable Evaporate

In some embodiments, said non-condensable evaporate, also referred to as a non-condensable fluid, can be further treated to remove substances such as dihydrogen, CO₂, ammonia or VFA that might be present in significant amounts.

Treatment of the non-condensable evaporate usually takes place in a scrubber, such as, but not limited to, spray towers or venturi scrubbers, membrane scrubbers, or other type of scrubbers evident to someone skilled in the art. In this scrubber a source of acid or base or solvent, or combinations thereof, might be required to recover the desired substances, for example, an acid would be used to recover ammonia, while a base is used to recover VFA. Alternatively, or in combination, some of the volatile compounds may be recovered using an appropriate solvent. A pH controller is usually part of these scrubber systems to maintain the pH of the scrubbing liquid inside the scrubber at a set pH level to optimize capture of the desired substance. The scrubber generally produces two main effluent streams: a product stream with the captured substance and the acid, base or a solvent, and a treated gas. Depending on the treatment performed, the treated gas may contain dihydrogen alone or mixed with other gases such as ammonia, VFA or CO₂. The treated gas may thus be reused in the process of the present disclosure for pH control and/or redox potential control of the bioreactor depending on its composition. Similarly, the product stream may be reused in the process for such controls.

### Optional Distillation of the Condensate

In one aspect of the disclosed embodiments, a distillation process is considered for the removal of one or several specific materials from the condensate, by any type of distillation process including, but not limited to, simple distillation (such as cooling in a condenser to a receiver vessel), short path distillation (short distance of travel to condenser), molecular distillation (where desired vapor molecules are forced into a condenser via a vacuum or drop in temperature and undesired molecules are eliminated by moving in opposite direction), fractional distillation (vapors are distilled many times for separation of components), thin-film distillation (such as in a heated cylinder with a blade and roller) or spinning band distillation (such as in a long column using a spinning band to force vapor generation and then converting it to liquid and to improve separation).

The distillate(s) may be reused in the process for pH control and/or redox potential control of the bioreactor depending on its composition. Such optional distillation is typically performed in a distillation equipment.

### DESCRIPTION OF THE DRAWINGS

The various disclosed embodiments will be better understood with reference to the figures, which show exemplary embodiments of the present disclosure.

FIG. 1 represents schematically a first embodiment of the system and method.

FIG. 2 represents schematically a second embodiment of the system and method.

FIG. 3A represents schematically a third embodiment of the system and method.

FIG. 3B represents schematically a fourth embodiment of the system and method.

FIG. 4 represents a graph of the HAC fraction and flash passes as a function of pH.

FIG. 5 represents schematically a fifth embodiment of the system.

On the figures, the same references designate the same elements.

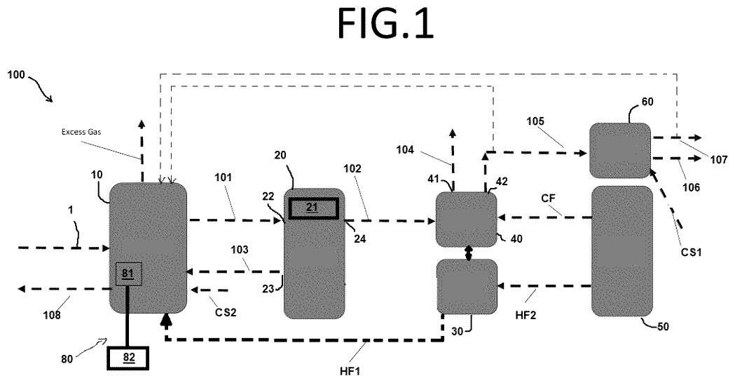

One embodiment of the process is presented in FIG. 1. A system 100 includes a bioreactor 10, here a fermenting bioreactor, a feed supply 1 to the bioreactor, an evaporator 20, typically comprising temperature and pressure control means 21 and pipes, valves, and pumps to fluidly connect the above-mentioned parts of the system. The bioreactor 10 is fluidly connected to an inlet 22 of the evaporator 20 and the evaporator 20 includes a first outlet 23 for a concentrate and a second outlet 24 for an evaporate, the first outlet 23 being fluidly connected to the bioreactor 10.

The temperature and pressure control means 21 of the evaporator 20 may include pump(s), valve(s), pressure gauge(s) and/or temperature sensor(s), and generally a programmable regulating unit (such as, but not limited to a microcontroller) for controlling such elements.

In one embodiment, the bioreactor 10 receives a feed source with carbonaceous material, such as, but not limited to, a slugged produced in the liquid treatment train of a wastewater treatment plant.

The bioreactor 10 is connected to an external evaporator 20, such as, but not limited to, a forced circulation evaporator, or a rising film or falling film evaporator, or an agitated thin film evaporator, or a multiple effect evaporator or a self-cleaning evaporator, or further a flash evaporator, also known to some as a flash cooler process, or others evident to someone skilled in the art.

The bioreactor and/or the evaporator 20 is connected to a source of heat 30, here a heat exchanger, that drives the evaporation process and/or drives the heating of the bioreactor, and the evaporator 20 is configured with a system (part of the control means 21) to limit and control the evaporation pressure and temperature inside the evaporator 20, for instance, a vacuum pump connected to its headspace. In some embodiments, where the evaporator 20 is a flash evaporator, the sensible heat of the liquid is used to drive the evaporation process that is conducted at a reduced, vacuum, pressure in the flash chamber. It is possible to send some of the volatiles removed from the evaporator 20 back to the evaporator in order to change or modulate pH or oxidation-reduction potential or to facilitate the volatilization, control stoichiometry or manage rates of reaction.

Temperature and pressure are controlled in the evaporator 20 to avoid damage to the microorganisms responsible for the catalytic activity in the bioreactor. Typically, the temperature in the evaporator is similar or lower than the temperature in the bioreactor. Some cooling of the aliquot 101 returned to the bioreactor usually takes place as a result of the evaporation process.

An aliquot 101 of the contents in the bioreactor 10, i.e. a portion of the broth contained in the bioreactor, is transferred to the external evaporator 20, where part of the water and other volatile substances such as, but not limited to, carbon dioxide, ammonia, volatile fatty acids, low molecular weight fermentation compounds, hydrogen sulfide or other odorous sulfur reduced compounds, are evaporated from the aliquot 101 forming an evaporate 102, and reducing the volume of the aliquot forming a concentrate 103. At least a fraction of the concentrate 103 containing the biocatalysts is returned to the bioreactor 10 where it is mixed with the contents of the bioreactor. The evaporate 102 containing water vapor, ammonia and other volatile substances, such as volatile fatty acids, is collected.

In some embodiments, as represented FIG. 1, the evaporate 102 is conducted to a condenser 40 where the evaporate temperature is reduced to the point where a fraction of the volatile molecules are condensed. The condenser thus includes a first outlet 41 for a condensate 104 and a second outlet 42 for a non-condensable fluid 105.

Yet in other embodiments, said evaporate is collected and treated to remove odors or recover non-condensable molecules such as CO₂, NH₃, H₂, CH₄.

In some embodiments the condenser 40 can be of the type that enables recovery of the heat of condensation using an equipment such as, but not limited to, the heat exchanger 30. The heat of condensation recovered HF1 can thus be provided to the bioreactor 10 and/or to the evaporator 20, preferably to the bioreactor as represented FIG. 1. In the latter case, another source of heat (not represented) may be connected to the evaporator if needed.

At least one source of cold fluid CF, here provided by, but not limited to, a heat pump 50, is connected to the condenser 40. The heat pump 50, in one embodiment, is connected to the heat exchanger 30 to provide heat HF2.

The evaporate that remains after passing through the condenser is the non-condensable evaporate 105. In the embodiment represented FIG. 1, the non-condensable evaporate 105, also referred to as a non-condensable fluid, is further treated to remove substances such as dihydrogen, CO₂, ammonia or VFA that might be present in significant amounts. Treatment of the non-condensable evaporate 105 here takes place in a scrubber 60 such as, but not limited to, spray towers or venturi scrubbers or other type of scrubbers evident to someone skilled in the art. In this scrubber system a source of chemical CS1 such as acid or base, or both, or a solvent, might be required to recover the desired substances, for example, an acid would be used to recover ammonia, while a base is used to recover VFA. A pH controller is usually part of such scrubber to maintain the pH of the scrubbing liquid inside the scrubber at a set pH level to optimize capture of the desired substance. Use of a solvent to recover substances is also possible. The scrubber 60 produces two main effluent streams: a product stream 106 with the captured substance and the acid, base, and/or the solvent, and a treated gas 107, containing the dihydrogen.

Three liquid streams could be removed from the system in FIG. 1, one is the condensate 104 produced in the condenser 40, the second one is a portion of the contents of the bioreactor 10 usually considered the bioreactor effluent 108, and a third is the product 106 from the scrubber 60 if a scrubber is used.

In some embodiments, including any of the herein described embodiments, the system 100 includes a parameter-control subsystem 80 of the bioreactor 10 that is electrically connected to the pumps that remove the aliquot 101 from the bioreactor 10 to the evaporator 20 and return the concentrate 103 to the bioreactor 10 forming an extraction loop. The frequency of removal and circulation of aliquot 101 and concentrate 103 is controlled by the parameter-control subsystem 80 to induce a variation of the pH in the bioreactor that would relief the inhibition associated with volatile fatty acids, dihydrogen and/or ammonia and/or to induce a variation in the redox potential that would select specific biochemical reactions. The parameter-control subsystem 80 is configured to control parameters of the bioreactor such as pH, redox potential. The parameter-control subsystem 80 may also be configured to control both parameters, or two parameter-control subsystems 80 may be provided, each one controlling a single parameter.

For example, once the increase in pH reaches a defined set point (corresponding to a higher limit of a set pH range) the parameter-control subsystem 80 would stop the circulation of the aliquot 101 to the evaporator 20 allowing for acids to accumulate and pH to depress; at determined set point of pH depression (corresponding to a lower limit of a set pH range) the parameter-control subsystem 80 will turn the circulation to the evaporator once more to remove the acids and increase pH. In this way a pH oscillation, up and down a set of selected set points, is created that relieve acid/dihydrogen inhibition in the bioreactor, enables acid/dihydrogen extraction and speeds the process.

In some other cases, no significant oscillation of pH is desirable, and the system is run in a more continuous basis to further enhance the extraction of acids. For example, the parameter-control subsystem 80 is configured to maintain the pH at a pH set point.

Yet in other embodiments, the parameter-control subsystem 80 may include, or consist of, a timer used to turn the pumps on and off to create the circulation of contents 101 of the bioreactor 10 to the evaporator 20 to remove acids/dihydrogen and to return the concentrate 103 to the bioreactor 10 in a specific cycle. When the pumps are off, acids accumulate decreasing pH while when the pumps are on extraction of acids takes place. The frequency and length of the intervals on and off can be calibrated to maximize the acid/dihydrogen formation and extraction.

In other embodiments the vacuum set point in the evaporator can be controlled to increase or decrease the efficiency of volatile substance stripping providing a way of achieving pH control. For example, the parameter-control subsystem 80 is then electrically connected to a pump that control the pressure inside the evaporator and is configured to reduce the vacuum inside the evaporator by controlling, eventually stopping, the pump.

Yet in other embodiments, the bioreactor 10 receives addition of chemical CS2 such as an acid, including, but not limited to, a CO₂ containing gas or VFA-containing gas, or a base including, but not limited to, CO₂ containing gas or ammonia-containing gas, to further decrease or increase and control the pH within the bioreactor 10 to enhance the extraction of volatile compounds in the evaporator. For example, an acid such as but not limited to hydrochloric acid, can be used to depress the pH to 5.5 and enhance the removal of VFA in the evaporator; while a base, such as, but not limited to calcium hydroxide, can be used to increase the pH to 8 or 9, or higher, to enhance the removal of ammonia in the evaporator 20. The acidification of the bioreactor may also be obtained by addition of a CO₂ containing gas or VFA-containing gas which is for example a portion of the treated gas 107 exiting the scrubber 60, or a portion of the non-condensable evaporate 105 leaving the condenser 40 as represented schematically on FIG. 1.

The addition of the acid or the base CS2 might be located directly in the bioreactor 10 or in the recirculation line to the evaporator or yet in other location to further enhance extraction.

Said addition might be part of the parameter-control subsystem 80 and operated continuously or intermittently to optimize the overall system performance. In such a case, the parameter-control subsystem 80 may include one or several capacities containing a base or an acid, and/or a supply for a CO₂ containing gas or any other gas, and pipes, valves and pumps to command the addition of the base, acid or CO₂ containing gas or any other gas in the system. The parameter-control subsystem 80 is then electrically connected to the pumps and/or valves. The parameter-control system also includes a parameter sensor 81 inside the bioreactor and one or more processors 82. The parameter sensor can be a pH sensor or a redox potential sensor, or the parameter control subsystem may include a pH sensor and a redox potential sensor.

External alkali or buffers can also be used including but not limited to caustic, lime, magnesia, potash, and the carbonates or bicarbonates of sodium, magnesium, calcium and potassium. Any salt of a strong base/weak acid can also be used.

The parameter-control subsystem as described in FIG. 1 can be an oxidation-reduction potential control subsystem to manage dihydrogen production and evaporation (in lieu of VFAs production and evaporation), and for example the addition of chemical CS2, can include an external or internal (by recycling) supply of dihydrogen, dioxygen or electric process (including but not limited to electrolysis, or the use of anodes or cathodes, or including but not limited to sacrificial anodes or cathodes). Instead of transitioning between pH values, the transitioning occurs between redox potential values as previously described.

A combination of pH- and oxidation-reduction potential-control subsystem is also possible, thus setting up a complex simultaneous or series approaches for removal. An artificial intelligence control system can be setup for removal of various volatile products.

The actual control can be either manual or on-line, with an analyzer located in a laboratory, in the bioreactor or evaporator, or handheld. The readings can be continuous, intermittent (the periodicity can vary in the order of seconds, minutes, hours or days). In many cases, the actual readings may be less important than the change in readings, such as between two setpoints, thus allowing for some drift of the analyzer. These can be assessed and compensated for using an artificial intelligence algorithm and underlying stochastics.

In one embodiment, combining the pH-and oxidation-reduction potential-control subsystems, the substrate for dihydrogen-producing anaerobic Clostridia is VFA, and the control of this unionized substrate using the pH-control influences the production of dihydrogen by managing substrate limitation, while the redox potential-control helps control the use of dihydrogen (by preventing methanogenesis if desired), as the increase in the oxidation-reduction potential improves hydrolysis, and the subsequent fermentative conversions to VAF, and further from VFA to dihydrogen. So, the combination of the two parameters-control subsystems can either maximize (by unbottlenecking VFA substrate and oxidation-reduction potential) or alternatively minimize dihydrogen production (by bottlenecking VFA substrate and oxidation-reduction potential) whichever is desired by controlling the two subsystems and the evaporation step. The evaporation of the dihydrogen or VFA removes the inhibitory effects of these volatile compounds. It should be noted that the combined use of at least two of the three concepts (pH-control, redox potential-control and evaporation) is envisioned in this embodiment as a means to select for desired reactions and deselect undesirable reactions. Thus, the approach proposed can eventually at steady state select for or deselect bacteria or archaea associated with reactions.

The above-described controls of the pH and/or redox potential can be applied in any embodiment of the system of the present disclosure.

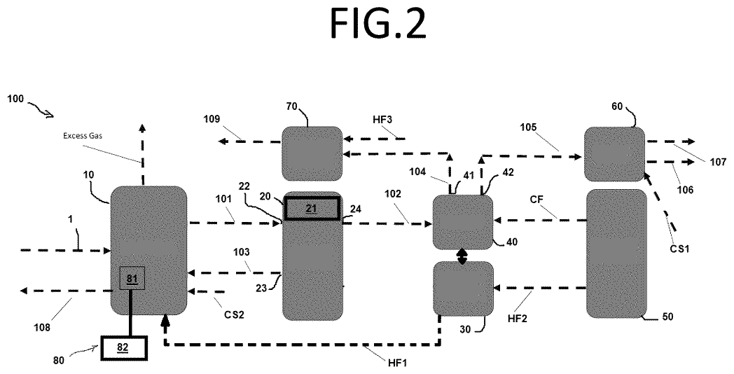

FIG. 2 illustrates one embodiment, as there could be others, of the system 100 where a distillation equipment 70 is used to further concentrate a specific target compound present in the condensate 104. FIG. 2 describes a system identical to FIG. 1 but with the addition of a distillation equipment 70 to receive the condensate and further process it. The same elements are designed by same reference numbers. Here the distillation equipment 70 receives heat HF3 to produce one or several distillates 109. The recycle of the condensate or treated gas described in reference to the FIG. 1 may be included in this embodiment.

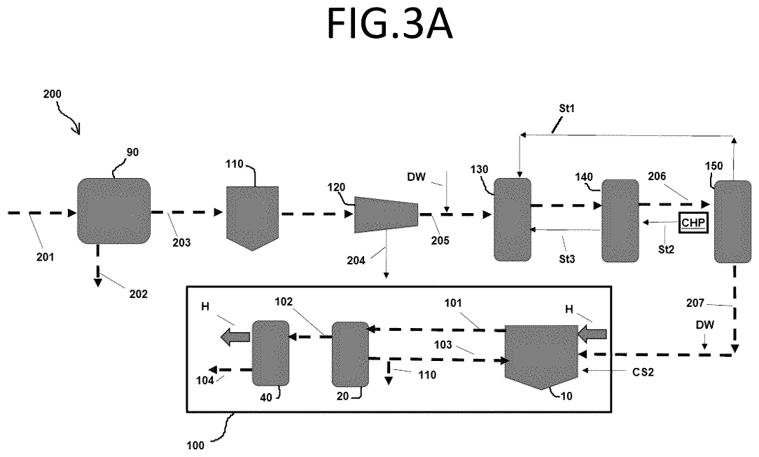

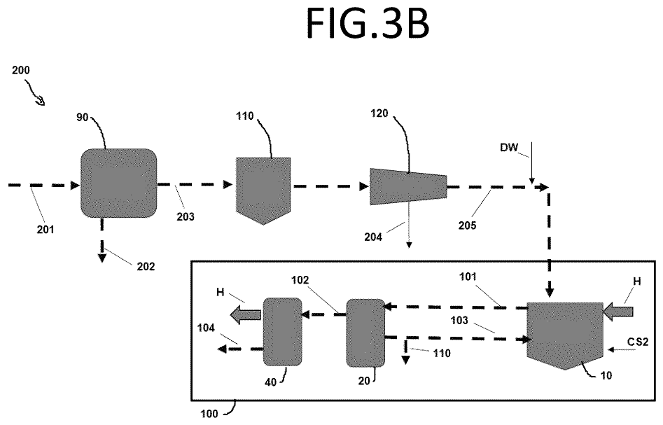

FIG. 3A illustrates one embodiment of the system 100 such as the one presented in FIG. 1 or FIG. 2 as part of the overall process flow diagram of sludge treatment in a wastewater treatment plant 200. This embodiment is illustrative in purpose as many variations of the location could be envisioned to someone skilled in the art, in particular the use of alternative thickening equipment, or screening equipment, or heat recovery and energy use optimization can have multiple arrangements, as well as addition of dilution water, to optimize viscosity, and use of different evaporation equipment, or thermal hydrolysis equipment. Several thermal hydrolysis processes exist in the market and the disclosed embodiments can be integrated with any of them. This example described in FIG. 3A does not preclude the application of the disclosed embodiments along other thermal hydrolysis processes.

In FIG. 3A thickened sludge 201 at a typical 2-15% concentration of dry solids (mass %), could be primary sludge produced during primary treatment of wastewater prior to a biological process or could be a waste biological sludge produced during secondary biological treatment such as waste activated sludge from an activated sludge process or could be a combination of thickened primary sludge and waste biological sludge. This sludge 201, in the embodiment presented in FIG. 3A, is screened in a solid screening process 90 to remove foreign objects that might interfere with the downstream processing. Said foreign objects removed are referred to as screenings 202 in FIG. 3A. The screened sludge 203 is conveyed to a blend tank 110 for storage and steady feeding to a dewatering equipment which in this case is a centrifuge 120, but other equipment could be used such as filtration equipment, forming two streams: a centrate stream 204 containing mostly water and a dewatered sludge stream 205, also referred to as a dewatered cake due to its consistency, with a typical concentration of solids between 15% and 30% (mass %). Dilution water DW is typically added at this stage to thin the sludge 205, although in some processes no dilution water is used.

In some embodiments, after addition of dilution water DW, the concentration of solids in the sludge conveyed to the pulper is about 17%-25% (mass %). A pulper 130 then receives the steam St1 from a flash tank 150 further contributing to the dilution of the sludge 205 with 15%-20% (mass %) dry solids content to preheat it and homogenize it. After the pulper 130, the sludge 205 is passed to one or several high-pressure reactors 140 where thermal hydrolysis takes place with the injection of steam St2 which, in this example, is produced in a combined heat and power unit, CHP, but could be steam St2 produced by other sources. Some of the excess steam St3 in the reactors 140 is directed towards the pulper 130 for preheating. After the thermal hydrolysis step in the reactors 140, the thermally treated sludge 206 is directed to a flash tank 150 where a sudden release of pressure takes place evaporating some of the water and disrupting the integrity of the sludge constituents further enhancing the hydrolysis of the sludge and forming a hydrolyzed sludge 207 with low viscosity. The evaporation that takes place in the flash tank 150 cools the sludge but depending on the process downstream additional cooling might be necessary. In the present embodiment, the hydrolyzed sludge 207 might be conveyed to a cooling process that could take place in a heat exchanger (not represented), or in a secondary flash process (not represented), but other means could be evident to someone skilled in the art. Depending on the overall heat balance of the system, the cooler might be bypassed, and the hydrolyzed sludge 207 conveyed directly to the bioreactor 10.

The low viscosity of the hydrolyzed sludge 207 is beneficial for the use of biological processes at high concentration of solids and a coupled evaporator. However, depending on the thermal hydrolysis process, additional dilution water DW might be added at this point to further the characteristics of the hydrolyzed sludge 207 to the requirements of the process downstream.

In the embodiment represented FIG. 3A, the hydrolyzed sludge 207 is conveyed to a system 100 such as the one previously described in FIG. 1 and FIG. 2, where formation of volatile fatty acids or ammonification of proteins or formation of dihydrogen is preferred for extraction in the evaporator. The same elements are designed by the same reference number. On FIG. 3A, “H” designs heat produced or received by an equipment. 110 designs spent sludge that may not be returned to the bioreactor and could be joined to the effluent leaving the bioreactor 10. As previously stated, the pH and/or redox potential of the bioreactor 10 can be controlled by parameter-control subsystem(s) (not represented here) for controlling the pH, the redox potential or both, to enhance either the removal of VFAs or ammonia or dihydrogen produced. For example, operating the bioreactor 10 at pH of 3 to 7 would induce better removal of VFAs in the evaporator 20, while operating at a pH of 8-10 would induce better removal of ammonia in the evaporator 20. In addition, operating between an oxidation-reduction potential value between +50 mV or −250 mV would induce better removal of hydrogen. As previously stated, FIG. 3A is illustrative in purpose and different ways of collecting and optimizing energy use can be used. Likewise different types of evaporation processes can be applied including but not limited to flash evaporation, or flash cooling, and the use of dilution water could be integrated as part of the process. The bioreactor 10 could also include a parameter-control subsystem as previously described to drive the evaporation process and optimize the extraction of VFA or ammonia. The process described in FIG. 3A include a thermal hydrolysis sub-step implemented by the elements 130, 140, 150. Such thermal hydrolysis sub-step may for example be implemented using the CAMBI® thermal hydrolysis process, however, other thermal hydrolysis processes exist that reduce the viscosity of the sludge to enable the use of evaporation of the liquid for extraction of volatile compounds, those thermal hydrolysis processes can also be coupled in alternative embodiments in the present disclosure.

FIG. 3B further illustrates an example of the use of this disclosed embodiment as part of a sludge processing train in a wastewater treatment plant 200 where the fermenting bioreactor 10 is operated at high concentrations of solids after dewatering. Other configurations are possible and someone skilled in the art can find variations to the processing train that best fit the local conditions. FIG. 3B describes a system identical to FIG. 3A but without the elements 130, 140, 150. The dewatered cake 205, optionally water diluted (DW), is here directly sent to the bioreactor 10. The evaporator 20 is particularly useful when operating a fermenting bioreactor 10 at high concentrations of solids because it enables at steady removal of fermentation products, such as VFA, that produce product inhibition in the fermentation reaction relieving the inhibition and enabling a more complete conversion. As previously stated, the pH and/or oxidation-reduction potential of the fermenting bioreactor 10 can be controlled using parameter-control subsystem(s) (not represented). As in previous applications, the evaporator 20 may be connected to a condenser 40 to recover the acids and heat that can be collected and recovered and reused in the fermentation process. Spent sludge 110 is collected and advantageously conveyed to downstream processing in an anaerobic digestion process, or in a thermal hydrolysis process followed by anaerobic digestion, to further stabilize the sludge and remove fermentation products that can interfere with final dewatering. Spent sludge 110 is typically treated with the effluent leaving the bioreactor 10.

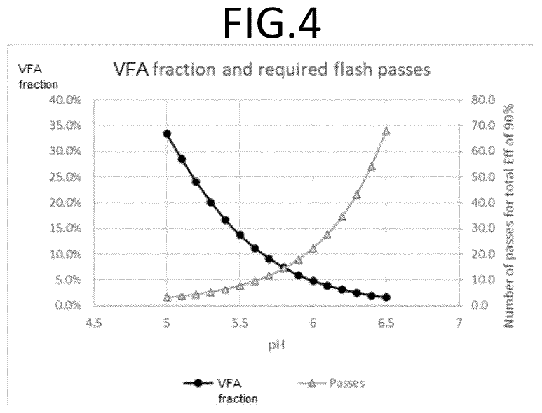

FIG. 4 illustrates the effect of operating the fermenter at a different pH on the efficiency of VFA removal in the evaporator. As the pH is reduced, the volatile fraction of the acids, named VFA fraction in the graph, increases and so the extraction efficiency in the evaporator. This is indicated by the number of required flash passes, or circulation, to obtain 90% (mass %) removal of the VFA in the fermenter liquor (fermenter broth) as it passes through the evaporator. It is clear from this graph that to enhance the efficiency of extraction operation at lower pH is beneficial.

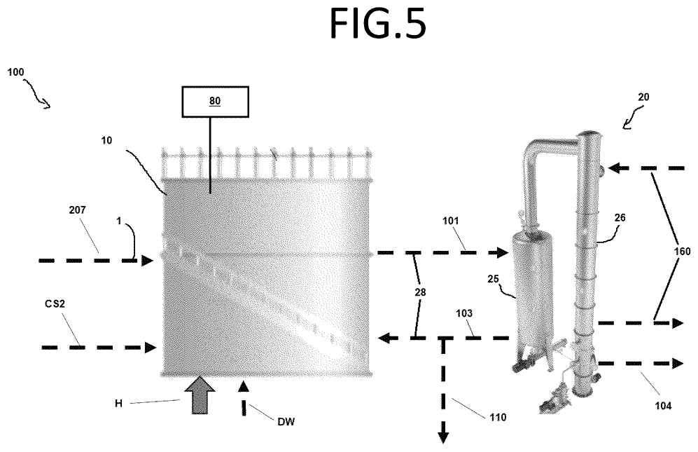

FIG. 5 illustrates one embodiment where the evaporator 20 is a flash evaporator, also marketed as a flash cooler, coupled with a fermenter 10 for extraction of VFA or ammonia or dihydrogen. The system 100 represented FIG. 5 may be used in any of the previous embodiments. In one embodiment, hydrolyzed sludge 207 from a thermal hydrolysis process is the input sludge 1 to the fermenter 10.

In one embodiment, the fermenter 10 is operated to a SRT of 0.5 day to 3 days. Yet, in other embodiments, the fermenter 10 is operated at a SRT of 4 days. The fermenter 10 is equipped with at least one parameter-control subsystem 80 to maintain the pH and/or oxidation-reduction potential of its contents to a defined set point, or within a range of pH or oxidation-reduction potential. Chemicals CS2, such as an acid (or CO2-rich treated gas, for example the treated gas from the scrubber 60 of FIG. 2, or the non-condensable evaporate) or a base may be added to the system as required for pH-control by the parameter-control subsystem to fit its purpose, and/or an oxidant or reductant as required for the oxidation-reduction potential control by the parameter-control subsystem to fit its purpose. For example, in one embodiment hydrochloric acid is added to bring the pH to a set point of about 5.5 units when the extraction of VFAs is desired. Yet, in other embodiment a base, such as calcium hydroxide is added to bring the pH to a set point of about 9 units when the extraction of ammonia is desired. Yet, in another embodiment, an oxidant such as air or dioxygen (O₂) is added to bring the oxidation-reduction potential up to 0 mV or higher or a reductant such as an electrochemical substance or dihydrogen is added to bring the oxidation-reduction potential down to −100 mV or lower. The contents of the fermenter 10 are circulated back and forth to the flash evaporator 20, also commercially available as a flash cooler, to flash evaporate water and volatile compounds under vacuum in a first chamber 25. The vapors are conducted to a second chamber 26 where cooling fluid is added to a shell and tube heat exchanger, forming a cooling loop 160, to induce condensation of vapors forming a condensate 104 containing the VFA or ammonia or dihydrogen depending on the pH and/or oxidation-reduction potential of the fermenter. The remaining liquid in the first chamber 25, the concentrate 103, is returned to the fermenter 10 to complete the extraction loop 28. Due to the cooling effect created by evaporation, the returned concentrate 103 is cooler than the feed to the flash chamber 25. The fermenter 10 is typically equipped with a temperature control system that would add heat to the unit as needed. In some embodiments the temperature of the fermenter 10 is controlled for mesophilic conditions (20-45° C.) while yet in other embodiments the temperature is controlled for thermophilic conditions (45-75° C.). Steam might be added for heat addition also providing some dilution water to maintain the viscosity of the fermenter contents. In other cases, heat H might be added using a heat exchanger coupled to the fermenter in which case dilution water DW might be necessary for viscosity control. The source of heat for the heat exchanger heating the bioreactor could be heat recovered using the heat pump or heat from a vapor recompression system, coupled to the evaporator 20. Excess sludge 110 is removed from the system at different locations, in one embodiment a fraction of the return concentrate from the flash chamber is removed.

In one aspect, applicable in any of the previous described embodiments, the vapor and/or condensate is stored as a chemical “on demand battery” to help promote downstream reactions within a dowstream reactor (such as a digester) to specifically generate desired products (such as dihydrogen or methane or any other compound or precipitate, on demand). This on demand battery could help with peak electrical load demands as a power/energy supply combined with a turbine or engine, hydrogen cell or a methane/natural gas grid network. The distilled or condensed product can be a raw material for any downstream industrial process (such as, but not limited to, food processing, plastics manufacturing or fertilizers), or consumer goods (such as, but not limited to, cleaners). The distilled or condensed product can also be used as a carbon source within a biological nutrient removal process (denitrification, deamonification, or biological phosphorus removal) or recovery process (such as, but not limited to, ammonium sulfate, struvite, brushite, vivianite), disinfection (peracetic or performic acid).

The hydrolysis process that could occur could be improved by adding chemicals, heat or electric input, including but not limited to acid, base, oxidant, reductant, e-beam, proton beam, thermal processes. The products generated by the process of the disclosed embodiments may be used in many applications—for example, household, municipal, agricultural or industrial applications—as cleaners, disinfectants, substrates for biological reactions, biofuels (including but not limited to biohydrogen, biomethanol, biomethane, or bioethanol), substrates for pH or redox management, etc.

Having thus described several aspects of at least one embodiment of disclosed embodiments, it is to be appreciated that various alterations, modifications, and improvements will readily occur to those skilled in the art. Such alterations, modifications, and improvements are intended to be part of this disclosure and are intended to be within the spirit and scope of the disclosed embodiments. Accordingly, the foregoing description and drawings are by way of example only.

The systems that are envisioned in the present disclosure include, but are not limited to, a heat pump(s), mechanical vapor recompression equipment, condenser(s), still(s) and associated equipment, vacuum, negative (such as including controlled or uncontrolled flash) or positive pressure (such as steam, venturi or a pump), or a force supply unit, external heat source (such as solar cells, chemicals, electricity, or batteries, or mechanical vapor recompression, or heat pump) or heat exchangers, hydrocyclones, screens or any separation device for two liquids or liquid and precipitates or other products generated, a source of alkali or acid and its mixing into the device (such as a pug mill or a mixer).

What is claimed is:

1. A method for treating a feed containing carbonaceous material in a bioreactor, the method comprising:
a) hydrolyzing the feed in the bioreactor using microorganisms and enzymes,
b) removing a portion of contents of the bioreactor, said removed portion containing microorganisms, enzymes and hydrolysis products, the hydrolysis products containing volatile inhibitory compounds,
c) evaporating a part of said removed portion and forming an evaporate containing at least a part of the volatile inhibitory compounds initially contained in said removed portion, and a concentrate containing at least the microorganisms initially contained in said removed portion,
d) returning at least a fraction of said concentrate to the bioreactor, and
e) collecting said evaporate.

2. The method of claim 1, further comprising:
condensing at least a fraction of the collected evaporate to form a condensate and a non-condensable evaporate.

3. The method of claim 2, further comprising:
(i) treating the non-condensable evaporate to remove at least one substance selected from dihydrogen, ammonia, CO₂, and volatile fatty acids, or (ii) submitting the condensate to a distillation to recover at least one distillate, or (i) and (ii).

4. The method of claim 2, further comprising:
using said condensate or said at least one distillate as a raw material for a chemical battery for a power or natural gas grid, biofuel, industrial, municipal, agricultural or household products, nutrient removal or recovery, or disinfection.

5. The method of claim 2, further comprising:
recovering heat of condensation for further use.

6. The method of claim 5, wherein the heat of condensation is recovered using a heat pump or a mechanical vapor recompression system or a heat exchanger.

7. The method according to claim 1, wherein at least one parameter, selected from a pH and an oxidation-reduction potential in the bioreactor, is controlled in step a):
by setting a setpoint range for said at least one parameter with a lower limit and an upper limit and activating steps b) to e) to maintain said at least one parameter within its setpoint range, or
by setting a setpoint value for said at least one parameter and continuously performing steps b) to e) to maintain said at least one parameter at its setpoint value, or
by setting activation periods at a specific activation frequency and activating steps b) to e) during a set duration at each activation period.

8. The method according to claim 7 wherein, during activation of steps b)-e), said at least one parameter is controlled in step a) (i) by adding at least one chemical selected from an acid, a base, an oxidant and a reductant (ii) by controlling a removal frequency of the portion removed from the bioreactor in step b), (iii) by controlling a flow rate of the portion removed from the bioreactor in step b), (iv) by controlling a temperature of the evaporation step c), (v) by controlling a pressure of the evaporation step c), or with a combination of one or several of (i) to (v).

9. The method according to claim 7, wherein, in step a), the pH in the bioreactor is controlled at a pH value from 3 to 7 or at a pH value from 8 to 10.5, and/or wherein the oxidation-reduction potential in the bioreactor is controlled at a value from +350 mV to −400 mV.

10. The method according to claim 1, further comprising at least one of the following steps:
providing a feed containing carbonaceous material containing a thermal treatment sub-step and submitting said feed to step a), and
submitting an effluent produced by the hydrolysis step a), and optionally the fraction of said concentrate not returned to the bioreactor, to a thermal treatment, and optionally to an anaerobic digestion process.

11. A system for treating a feed containing carbonaceous material in a bioreactor, the system comprising:
the bioreactor, a feed supply to the bioreactor, an evaporator, and pipes, valves, and pumps to fluidly connect the above-mentioned parts of the system, wherein said bioreactor is fluidly connected to an inlet of the evaporator, and wherein said evaporator comprising a first outlet for a concentrate and a second outlet for an evaporate, the first outlet being fluidly connected to said bioreactor.

12. The system of claim 11 further comprising: a condenser, wherein at least one source of cold fluid is connected to the condenser, the condenser being fluidly connected to the second outlet of the evaporator, and the condenser comprising a first outlet for a condensate and a second outlet for a non-condensable fluid.

13. The system of claim 12 further comprising: at least one of the following features:
(i) a scrubber and a source of at least one chemical selected from an acid, a base or a solvent fluidly connected to said scrubber, and said scrubber being fluidly connected to the second outlet of the condenser, and
(ii) a distillation equipment connected to the first outlet of the condenser.

14. The system of claim 12 further comprising: a heat recovery equipment selected from a heat pump, a heat exchanger, a mechanical vapor recompression system, said heat recovery equipment being connected to the condenser to recover the heat of condensation and connected to the bioreactor or to the evaporator to provide heat thereto.

15. The system of claim 11 further comprising: at least one parameter-control subsystem for controlling at least one parameter of the bioreactor selected from a pH and an oxidation-reduction potential in the bioreactor, said subsystem being configured to:
set a setpoint range for said at least one parameter with a lower limit and an upper limit and perform the following commands to maintain said at least one parameter within its setpoint range:
C1) removing a portion of contents of the bioreactor and introducing said removed portion into the evaporator, said removed portion containing microorganisms, enzymes and hydrolysis products, the hydrolysis products containing volatile inhibitory compounds,
C2) evaporating a part of said removed portion to form an evaporate containing at least a part of the volatile inhibitory compounds initially contained in said removed portion, and a concentrate containing at least the microorganisms initially contained in said removed portion,
C3) returning at least a fraction of said concentrate to the bioreactor and,
C4) collecting said evaporate,
or
set a setpoint value for said at least one parameter and continuously perform commands C1) to C4) to maintain the said at least one parameter at its setpoint value,
or
set activation periods at a specific activation frequency and perform commands C1) to C4) during a set duration at each activation period.

16. The system of claim 15, wherein the parameter-control subsystem is further configured to, during the performing of commands C1) to C4): control said at least one parameter in the bioreactor (i) by adding at least one chemical selected from an acid, a base, an oxidant, a reductant, (ii) by controlling a removal frequency of said portion removed from the bioreactor, (iii) by controlling a flow rate of said portion removed from the bioreactor, (iv) by controlling a temperature of the evaporator, (v) by controlling a pressure of the evaporator, or with a combination of one or several of (i) to (v).

17. The method of claim 5, wherein the recovering of the heat of condensation is used for the evaporating step c) or for the hydrolysis step a).
